(19) United States
(12) Patent Application Publication
(10) Pub. No.: US 2026/0007902 A1
(43) Pub. Date: Jan. 8, 2026

(54) SYSTEM AND METHOD FOR LASER HAIR TREATMENT

(71) Applicant: LUMENIS BE LTD., YOKNEAM (IL)

(72) Inventors: Amir Kishon, Yokneam (IL); Sachi Norman, Yokneam (IL); Zoya Cahana, Yokneam (IL); Karin Srage, Yokneam (IL); Assaf Preiss, Shimshit (IL); Moshe Elazar, Kadima-Tzoran (IL); Kalia Mendel, Yokneam (IL)

(21) Appl. No.: 19/318,954

(22) Filed: Sep. 4, 2025

Related U.S. Application Data
(63) Continuation-in-part of application No. 18/648,431, filed on Apr. 28, 2024.
(60) Provisional application No. 63/463,969, filed on May 4, 2023.

(51) Int. Cl. A61N 5/06; A61N 5/067

(52) U.S. Cl. CPC A61N 5/0617; A61N 5/067; A61N 2005/0627; A61N 2005/0632; A61N 2005/0642; A61N 2005/0644

(57) ABSTRACT

A method and system for hair treatment comprising a laser module configured for generating laser light; a handpiece connected to the laser module configured to receive the laser light and deliver a fractional pattern of non-ablative laser beams to a treatment site on a person's scalp; a controller for controlling parameters of the non-ablative laser beams to apply the hair and activating the laser module for a predetermined treatment time, at a predetermined pulse rate, and a predetermined pulse duration; delivering, by the handpiece, the non-ablative laser to the treatment site on the person's scalp; and deactivating, by the controller, the laser module.

### RELATED APPLICATIONS

This application is a continuation-in-part, and claims the priority benefit, of U.S. application Ser. No. 18/648,431 filed Apr. 28, 2024, entitled “System and Method for Laser Hair Treatment,” the entire contents of which are herein incorporated by reference; which in turn claims the priority benefit of U.S. Provisional Application No. 63/463,969 filed May 4, 2023, entitled “System and Method for Laser Hair Treatment,” the entire contents of which are herein incorporated by reference.

### TECHNOLOGICAL FIELD

The present invention is in the aesthetic treatment field and relates specifically to hair treatment, and more particularly to improving scalp hair condition.

### BACKGROUND

Hair loss, or alopecia, is a major problem that affects the majority of the population, both men and women, at some point in their life, and can be caused by a variety of conditions. Alopecia is defined as a common condition in which the hair fails to adequately cover the scalp, and thus, can cause psychosocial distress.

Common baldness, otherwise known as androgenic alopecia, is the most common type of non-scarring alopecia, and is characterized by the miniaturization of hair follicles (HF), shortened anagen and prolonged telogen hair growth cycle phases.

Numerous treatment solutions are on the market, including non-invasive and invasive treatments. The invasive treatments include surgical procedures which can be accompanied with pain and inflammation risks. The non-invasive or minimally invasive treatments include, for example, injectables, topical treatments, supplement oral treatments and treatments by energy, such as optical energy. Topical corticosteroids are often the first line of treatment for mild patchy alopecia. However, this method cannot be used for rapidly progressing variants and does not prevent hair loss at other sites. Furthermore, both topical and intralesional steroids increase the risk of cutaneous atrophy at the site of treatment, and intralesional steroids may decrease bone mineral density. Systemic corticosteroids have also had promising results, with one study demonstrating 62% of patients exhibiting full hair regrowth. However, the therapy can cause substantial adverse side effects such as acne, myalgias, osteoporosis, numbness, and weight gain.

### SUMMARY OF INVENTION

In at least one aspect of the invention, there is a method for hair treatment comprising: providing a laser module configured for generating a non-ablative laser beam; providing a handpiece connected to the laser module configured to receive the non-ablative laser beam, and deliver a fractional pattern of the non-ablative laser beams to a treatment site on a person's scalp; providing a controller adapted for controlling parameters of the non-ablative laser beam to apply the hair; activating, by the controller, the laser module for a predetermined treatment time, at a predetermined pulse rate, and a predetermined pulse duration; delivering, by the handpiece, the non-ablative laser beams to the treatment site on the person's scalp; and deactivating, by the controller, the laser module.

In another aspect, there is a system for hair treatment comprising a laser module configured for generating laser light; a handpiece connected to the laser module, configured to receive the laser light and deliver a fractional pattern of non-ablative laser beams to a treatment site on a person's scalp; one more of tips, each comprising a treatment window, configured to be mounted at a distal end of the handpiece; a controller adapted for controlling parameters of the non-ablative laser beams to apply to the person's scalp; a non-transitory computer readable medium programmed with computer readable code that upon execution by the controller causes the controller to: activate the laser module for a predetermined treatment time, at a predetermined pulse rate and a predetermined pulse duration; deliver the non-ablative laser beams to the treatment site on the person's scalp; and deactivate the laser module.

In some embodiments of the system, the treatment window is made from sapphire.

In some embodiments, the treatment window has a shape of at least one of the following: a polygonal shape; a non-regular shape; a half polygon shape; a half circle shape; a circle shape: a half elliptical shape; an elliptical shape; and a U shape.

In some embodiments of the system, the tip comprises a visualization window configured to provide visualization of the treatment site and/or the treatment window.

In some embodiments of the system, the visualization window is a hole or opening in a wall of the tip.

In some embodiments of the system, wherein the tip comprises a plurality of visualization windows with at least one of a front side orientation and a back side orientation, in relation to a treatment direction.

In some embodiments of the system, the tip is either a disposable tip configured for a single use or a single patient; or a reusable tip adapted for sterilization between treatments.

In some embodiments of the system, at least one of the one or more tips further comprises an elongated bar forming a spacer defined by a distance between the laser output and the treatment site.

In some embodiments of the system, the spacer is at least one of: an integral part of the tip or a connectable spacer to a distal end of the tip.

In some embodiments of the system, the one or more tips are configured to be mounted at a distal end of the handpiece, and the tips are configured to do at least one of the following: define the shape of the treatment site; form a spacer between a laser output of the handpiece and the treatment site; move grown hair aside for treatment; or conform to topography of the treatment site.

In another aspect, there is method for hair treatment comprising: providing a laser module configured for generating laser light; providing a handpiece connected to the laser module configured to receive the laser light and deliver a fractional pattern of non-ablative laser beams to a treatment site on a person's scalp; providing one more of tips, each comprising a treatment window, configured to be mounted at a distal end of the handpiece; providing a controller adapted for controlling parameters of the non-ablative laser beams to apply the scalp; activating, by the controller, the laser module for a predetermined treatment time, at a predetermined pulse rate and a predetermined pulse duration; delivering, by the handpiece, through the treatment window, the non-ablative laser beams to the treatment site on the person's scalp; and deactivating, by the controller, the laser module.

In some embodiments of the method, the treatment window is made from sapphire.

In some embodiments of the method, the tip comprises a visualization window configured to provide visualization of the treatment site and/or the treatment window.

In some embodiments of the method, the visualization window is a hole or opening in a wall of the tip.

In some embodiments of the method, the tip comprises a plurality of visualization windows with at least one of a front side orientation and a back side orientation in relation to a treatment direction.

In some embodiments of the method, at least one of the one or more tips further comprises an elongated bar forming a spacer defined by a distance between the laser output and the treatment site.

In some embodiments of the method, the spacer is at least one of an integral part of the tip or a connectable spacer to a distal part of the tip.

In some embodiments of the method, the delivering, by the handpiece, of the non-ablative laser beams to the treatment site on the person's scalp is done by sliding or gliding the tip over the treatment of the person's scalp.

### BRIEF DESCRIPTION OF THE DRAWINGS

FIG. 1 illustrates a diagram of a system of at least some embodiments of the present disclosure.

FIGS. 2A1 to 2F3 illustrates multiple embodiments of a tip for the system of at least some embodiments of the present disclosure.

FIG. 3 is a flow diagram of the method of at least some embodiments of the present disclosure.

FIG. 4 illustrates the regeneration of hair follicles under treatment(s) carried out with the presently disclosed subject matter of at least some embodiments of the present disclosure.

### GENERAL DESCRIPTION

The presently disclosed subject matter relates to non-invasive systems and methods for hair treatment, specifically improvement of scalp hair appearance through, inter alia, new hair growth on the scalp and/or reducing or preventing hair loss. A fractional laser treatment applied to the scalp causes localized tissue coagulation, triggering a wound healing response that recruits the body's mechanisms of cell growth to regenerate the skin and adnexal structures such as hair follicles. In some embodiments, treatments are provided with lasers and monochromatic light, for example, with the use of a 308-nm excimer laser, a 904-nm pulsed infrared diode laser, or with a neodymium-doped yttrium aluminum garnet (Nd: YAG) laser to treat alopecia are employed.

To better understand the subject matter that is disclosed herein and to exemplify how it may be carried out in practice, embodiments will now be described, by way of non-limiting examples only, with reference to the accompanying drawings.

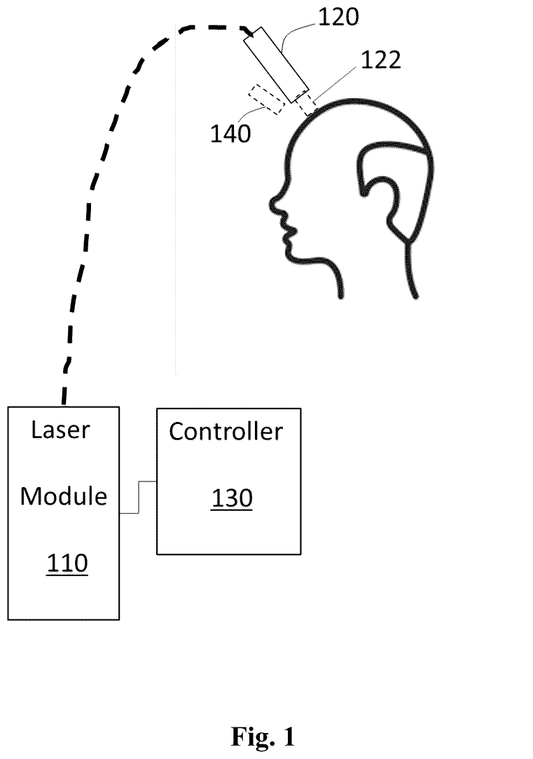

Reference is made to FIG. 1 illustrating, by way of a block diagram, a non-limiting exemplary embodiment of a system 100 according to the presently disclosed subject matter. The system 100 is operable for applying treatment to improve appearance of scalp hair, e.g. by promoting hair growth and/or reducing hair loss. The system includes a laser module 110 configured and operable to generate laser light for use in the hair treatment. A handpiece 120 is in optical communication with the laser module, either being directly connected to the laser module or via an optical fiber and configured to receive the laser light and deliver one or more laser beams to a treatment site on a subject's scalp. The handpiece may be adapted to deliver a fractional pattern of laser beams to a treatment site on a person's scalp. The delivered laser beams are non-ablative. In some embodiments, a controller 130 is provided in communication with the laser module and the handpiece. The controller 130 may be configured to control one or more parameters of the laser module or/and the handpiece to apply the hair treatment, thereby improving appearance of the hair. In some embodiments, the system includes non-transitory computer readable medium (not shown) programmed with computer readable code.

In some embodiments, the laser module 110 of the system 100 is configured for generating laser light having a wavelength in the range of 1000-2000 nm. In some embodiments, the laser module 110 is configured for generating laser light having at least one of the following wavelengths (in nm): 1940, 1927, 1565, 1550, 1540, 1470, 1450, 1440, 1410, 1340, 1320, 1064 and 1060. In some embodiments, the laser module 110 utilizes an erbium-dobed glass (Er: glass) fiber laser. In some embodiments, the laser module 110 utilizes a Nd: YAG laser.

In some embodiments, the handpiece 120 (or a probe, that can be operated by hand or by electrical or mechanical means) of the system 100 is adapted to deliver the laser beam as a fractional pattern of non-ablative laser beams comprising a plurality of spaced-apart micro-beams. The micro-beams may be formed to a certain shape defining the treatment site. In some embodiments, the micro-beams are patterned to form at least one of the following shapes: hexagon, annulus, circle, square, rectangle, and line. In some embodiments, the handpiece of the system is adapted to deliver the fractional pattern of non-ablative laser comprising a plurality of spaced-apart micro-beams forming a circular shape having a diameter of up to 18 mm defining the treatment site.

In some embodiments, the handpiece of the system is configured to deliver the fractional pattern of a non-ablative laser beams comprising a plurality of spaced-apart micro-beams having a density in a range of 50 to 500 micro-beams per cm². In some embodiments, the laser module system is part of a skin diagnostic and treatment system using machine learning models. In some embodiments, the skin diagnostic and treatment system analyzes captured multi-spectral images to automatically determine the treatment parameters of the laser module system, including a customized fractional pattern of spaced-apart micro-beams following, for example, the shape of the area of scalp lacking hair. In some embodiments, the skin diagnostic and treatment system analyzes a set of multi-spectral images taken right after the laser module system treatment to determine new treatment parameter and a customized fractional pattern for the laser module system.

In some embodiments, the system 100 is adapted to deliver the non-ablative laser micro-beams in pulses. In some embodiments, the pulses have a pulse duration of up to 10 ms. In some embodiments, the pulses have a pulse rate of between 0.5 to 2 Hz. In some embodiments, the system 100 comprises a registration module (possibly within the controller 130) configured to register locations of treatment sites visited during a treatment session, to enable guidance to the same treatment sites during a subsequent treatment session.

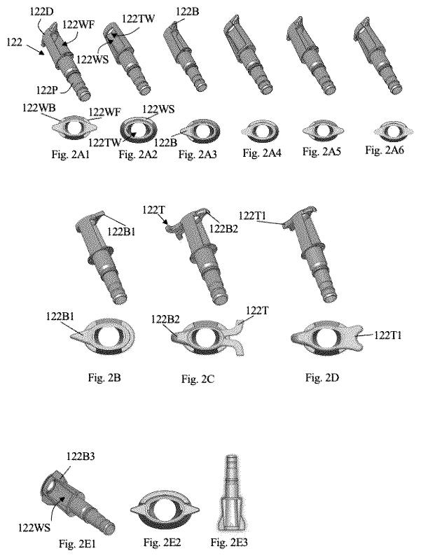

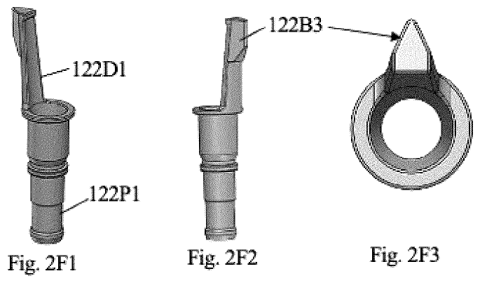

In some embodiments, the system includes one or more tips 122, mountable on the distal end of the handpiece. More details on tips 122 are illustrated in FIGS. 2A-2F3.

In some embodiments the one or more tips 122 define respective treatment site area or/and shape. In some embodiments, the one or more tips 122 form a spacer between a laser output of the handpiece and the treatment site. In some embodiments, the one or more tips 122 are configured to clear a path of the non-ablative laser micro-beams towards the treatment site on the scalp by moving aside grown hair. In some embodiments, the surfaces of the distal ends 122D of the one or more tips 122 are shaped to conform to topography of the treatment site.

In some embodiments, the system 100 includes one or more cooling devices 140. The cooling device(s) may be provided within the handpiece 120 or be provided in a separate enclosure. In some embodiments, the handpiece 120 comprises a first cooling device configured to maintain temperature of the handpiece or/and a tip mounted on a distal end thereof at an optimal temperature. In some embodiments, the optimal temperature of the tip is in the range of 7-13° C. The treatment site can be passively cooled by the tip contacting the treatment site. In some embodiments, the system comprises a second cooling device configured to cool the treatment site and keep it at an optimal temperature. Cooling the treatment site may aid in reducing or eliminating pain that might be caused by the treatment. Either the first or the second cooling devices, or both, may be provided in the system.

In some embodiments, the controller 130 is configured to automatically activate the first and/or second cooling device when the controller detects certain energy level and/or density level above predetermined or undetermined levels. In some embodiments, the controller 130 is configured to gradually increase the cooling intensity upon corresponding gradual increase in the energy level and/or the density level. In some embodiments, the controller 130 is configured to automatically activate the first and/or second cooling device 140 based on feedback from one or more temperature sensors reporting temperature of the handpiece 120 and/or the tip 122 and/or the treatment site to the controller.

FIGS. 2A1-2F3 illustrate non-limiting examples of tips 122 for mounting on the distal end of the handpiece for delivering the fractional non-ablative laser treatment to the scalp.

FIGS. 2A1-2A6 show isometric and bottom views of respectively six non-limiting possible configurations of tips having one or more features, as will be described below.

As shown in FIG. 2A1, each tip 122 has a proximal side 122P for connecting the tip 122 to the handpiece 120, and a distal side 122D which faces the treatment site. The distal side includes a treatment window 122TW that is transparent to the treatment laser light. In some embodiments, the treatment window 122TW is made from sapphire.

The treatment window 122TW may have a shape and a size adapted to the treatment site. In some embodiments, the treatment window 122TW allows delivery of the non-ablative laser micro-beams to the treatment site. In some embodiments, the treatment window 122TW has a circular shape, a polygonal shape (e.g. rectangular, hexagonal), or an oval/elliptic shape. In some embodiments, the treatment window 122TW has a non-regular shape such as half polygon, half round (circle or ellipse), or U shape.

The distal ends 122D of the tips 122 in FIGS. 2A1-2E2 are exemplified with an oval/elliptic shape that optimizes operation when passing the end 122D over the scalp. In some embodiments, a hair pass is created when passing the tip over the scalp. The hair pass is a treatment pathway in which a practitioner displaces the hair to expose the scalp surface for direct therapeutic application, such as when the tip 122 is slid or glided over a treatment area. The elliptic shape of the treatment window enables the tip to get close to the scalp between hair, thus enabling precise localization of the tip.

In some embodiments, the tip includes a visualization window 122WF, 122WB, 122WS (collectively, 122W) that provides or enhances visualization for the treatment area through the treatment window.

The visualization window 122W may be formed as a hole/opening in the wall of the tip. As shown, the tip 122 may include one or more visualization windows 122W that enable viewing the target treatment area from one or more different positions or directions. A plurality of windows may be advantageous for the user/operator when positioned at different sides with respect to the treatment area. As exemplified, the tip may include one or more of the following: a front visualization window 122WF and/or a back visualization window 122WB, that enable visualization along the treatment path/hair pass. Additionally, or alternatively, a left and/or right-side visualization window (only left window 122WS is shown) providing a side view with respect to the treatment path, can be included in the tip.

FIG. 2A1 illustrates two visualization windows 122W: both front and back visualization windows (back visualization window not shown). FIGS. 2A2, 2A4 and 2A6 illustrate one-sided visualization windows (left or right). FIGS. 2A3 and 2A5 illustrate a back or front visualization window (depending on the progression direction of the tip).

In some embodiments, the tip 122 includes, at the distal end 122D, a hair separator or bow protrusion 122B that is configured to facilitate advancing the tip end 122D through the hair, through a series of treatment areas and creating a hair pass. In some embodiments, the hair separator 122B engages with the treatment site, and is configured for spreading/parting the hair and creating a hair treatment path in the treatment site. The hair separator 122B may be in the form of a lateral protrusion/projection from the front edge of the end 122D of the tip 122. In some embodiments, the tip 122 comprises a single hair separator 122B protruding from the front edge of the tip end 122D. In some embodiments, the tip 122 comprises two hair separators 122B, at the front and back edges of the tip end 122D.

In some embodiments, a single hair separator 122B is provided in the tip 122, at the front edge of the tip end 122D. In this case, the tip may be unidirectional, progressed through the treatment area in one direction. Alternatively, providing a tip 122 with a single hair separator 122B enables optional use of the hair separator 122B, i.e. it is up to the operator to progress the tip in the direction of the hair separator 122B or in the opposite direction that lacks a hair separators. Examples of single-hair separator tips can be seen in FIGS. 2A3 and 2A4.

In some embodiments, two hair separators 122B are provided, at the front and back edges of the tip end 122D, and the tip 122 is bidirectional. The dual hair separators configuration may be advantageous with different operator positions relative to the treatment area. Examples for tips 122 with two hair separators 122B can be seen in FIGS. 2A1, 2A5, and 2A6.

In some embodiments, the hair separator is curved upwardly at its front, such as hair separators 122B1 or 122B2, shown in FIGS. 2B and 2C. A curved hair separator 122B1, 122B2 may be more effective in spreading the hair and creating the hair pass, in at least some treatment regions.

The hair separators(s) can enhance the cooling of the tissue during treatment. A back hair separator, in particular, may be effective in cooling/soothing the tissue after it was treated with the laser through the treatment window.

FIGS. 2E1-2E3 illustrate a tip with a side visualization window 122WS, elliptical treatment window 122TW and two hair separators 122B3 (that can function also as a hair separator and a tail as described herein). The hair separators 122B3 have an extended depth, protruding from the tip wall near the tip end 122D, towards the proximal side of the tip, and the hair separators 122B3 have a wing-like shape. During treatment, after a hair pass/line/path is created, the tip 122 is moved along the hair path. The wing-like front hair separator 122B3 assists in keeping the path effectively open for lasing by pushing the hair away along the direction of treatment and preventing hair (particularly long hair) from entering the path, and the wing-like back hair separator 122B3, which may have the same shape as the front hair separator, assists in dragging less hair from the area already treated.

In some embodiments, the tip 122 includes a tail 122T located at the back edge of the tip end 122D and configured to keep hair (especially short hair) away from the treated area, during the treatment, and/or support returning of the hair to position, shortly after the treatment, and/or aid in cooling the treated tissue (like a back hair separator). FIG. 2C shows a hook tail 122T and FIG. 2D shows an iron tail 122T1. In some embodiments, as readily appreciated, the tip end includes a hair separator on the front edge and a tail on the back edge.

FIGS. 2F1-2F3 illustrate a non-limiting example of a tip having a proximal side 122P1 for connecting the tip to the handpiece, and a distal side 122D1 in the form of an elongated bar forming a spacer defining a distance between the laser output and the treatment site and/or an irradiation orientation/alignment definer, which faces and engages with the treatment site. The spacer/aligner may be integral with the tip body or may be connectable to the distal end of the tip, enabling using different spacers defining respective distances between the laser output and the treatment site. As shown, the elongated bar 122D1 also includes a hair separator 122B3, aiding in creating a treatment path between separated hair. It is appreciated that in this example, the tip does not provide cooling to the treated tissue as in the previously described tips. In such case, cooling may be provided by the cooling device 140 described herein.

In some embodiments, the treatment window 122TW, the hair separator 122B and/or the tail 122T of the tip end 122D provide cooling of the treatment site, either by passively cooling the treatment site or by being actively cooled and transferring the cooling to the treatment site. In some embodiments, the tip is 122 uncooled and the cooling may be provided through another device.

In some embodiments, the tip 122 is disposable and adapted for a single use or a single patient. In some embodiments, the tip 122 is reusable and adapted for sterilization between different treatments.

In some embodiments, the tip 122 and the handpiece 120 are configured, when attached together, to enable rotation of the tip about its longitudinal axis. In some embodiments, the tip alone is rotatable around a longitudinal axis thereof.

In some embodiments, the handpiece 120 comprises a scanner for defining at least one of size and shape of the fractional pattern output of the laser beams. In some embodiments, the scanner is a galvanometric scanner.

In some embodiments, the method used in the system comprises irradiating a target portion on the scalp with a fractional non-ablative laser having a wavelength of 1565 nm, the fractional non-ablative laser beams comprising a plurality of spaced-apart micro-beams having a density in a range of between 50 and 500 micro-beams per cm² and an energy of between 10-35 mJ per each micro-beam. In some embodiments, the energy is between 5-70 mJ per each micro-beam. In some embodiments the energy is between 15-20 mJ per each micro-beam.

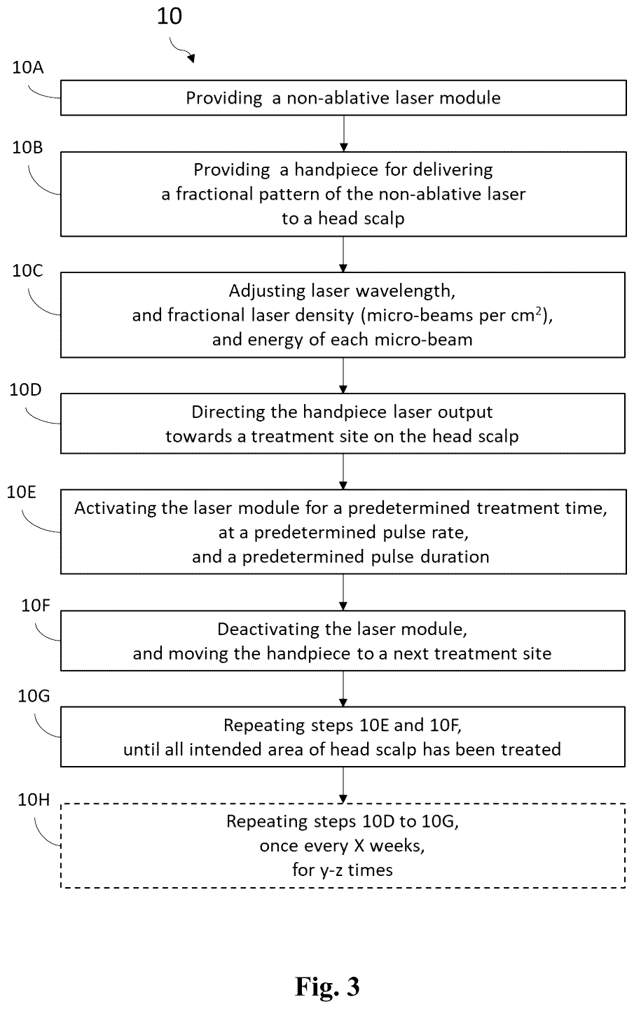

FIG. 3 illustrates, in a way of flow diagram, a non-limiting exemplary embodiment of a method 10 according to the presently disclosed subject matter.

At step 10A, the method comprises providing a laser module.

At step 10B, the method comprises providing a handpiece for delivering a fractional pattern of non-ablative laser beams to a head scalp. In some embodiments, the step includes providing a tip mounted on the distal end of the handpiece, as described above. In some embodiments, the method comprises providing a tip for the handpiece comprising a treatment window through which the non-ablative laser beams are delivered to the treatment site. In some embodiments, the treatment window is made from sapphire. In some embodiments, the treatment window has a polygonal shape. In some embodiments, the treatment window has an elliptical shape. In some embodiments, the treatment window has a non-regular shape such as half polygon, half round (circle or ellipse), U shape.

In some embodiments, the method comprises providing a tip comprising a visualization window configured to provide visualization of the treatment site and/or the treatment window. The visualization window may be formed as a hole/opening in the wall of the tip. In some embodiments, the method comprises providing a tip comprising a single visualization window. In some embodiments, the method comprises providing a tip comprising a plurality of visualization windows enabling visualizing the treatment site and/or treatment window from different operator positions. In some embodiments, the method comprises providing a tip comprising a front visualization window. In some embodiments, the method comprises providing a tip comprising a back visualization window. In some embodiments, the method comprises providing a tip comprising a side visualization window. The front, back and side orientations refer to the treatment direction: the “front” is facing the direction movement of the handpiece.

At step 10C, the method comprises adjusting the laser wavelength of the laser module (e.g. 1565 nm), and fractional laser density (e.g. 350 micro-beams per cm²), and energy of each micro-beam (e.g. 17-19 mJ per micro-beam).

At step 10D, the method comprises directing the handpiece laser output towards a treatment site on the head scalp. In some embodiments, the method comprises providing a tip comprising, at a distal end that engages with the treatment site, a hair separator configured for supporting advancing the tip end through the hair, spreading the hair and creating a hair treatment path in the treatment site. The hair separator may be in the form of a lateral protrusion/projection from the wall of the tip end. In some embodiments, the method comprises providing a tip comprising a single hair separator at the front side of the tip. In some embodiments, the method comprises providing a tip comprising two hair separators, at the front and back sides of the tip. In some embodiments, the method comprises providing a tip comprising the hair separator being upwardly curved at its front side to enhance the hair spreading along the progression direction of the tip. In some embodiments, the method comprises providing a tip comprising the hair separator(s) having an extended height, proximally along the wall of the tip end, having a form of a wing-like structure. In some embodiments, the method comprises providing a tip comprising the hair separator(s) having a sharp front edge.

In some embodiments, the method comprises providing a tip comprising, at a distal end that engages with the treatment site, a tail (at the back side of the tip end) configured to keep hair away from the treatment site during treatment and/or facilitate returning the hair to the original position after the treatment. In some embodiments, the method comprises providing a tip comprising a tail having a hook tail or an iron tail configuration. In some embodiments, the method comprises providing a tip that includes a bow on the front side of the tip end and a tail on the back side of the tip end. In some embodiments, when the provided tip includes two bows, a front bow functions as a bow, as described above, and a back bow functions as a tail, as described above.

At step 10E, the method comprises activating the laser module for a predetermined treatment time, at a predetermined pulse rate (e.g. 0.5-2 Hz), and a predetermined pulse duration (e.g. 10 ms for each pulse).

At step 10F, the method comprises deactivating the laser module, and moving the handpiece to a next treatment site, which can be adjacent to or located at a predetermined location with respect to the previous treatment site. In some embodiments, the treatment route is planned to minimize pain resulting from the treatment. For example, the treatment stamping route can be random or semi-random. In some embodiments, the fractional pattern of the laser can be a line or narrow rectangle, for example at an area with grown hair, or rectangular/circular/hexagonal, at areas lacking grown hair.

At step 10G, the method comprises repeating steps 10E and 10F, until all of the intended area of head scalp has been treated.

At step 10H, the method comprises repeating steps 10D to 10G, every X (e.g. 2-4) weeks, for y-z (3-6) times. In some embodiments, the method comprises a registration process recording the e previous treatment sites visited in a treatment session during step 10E, to serve as guidance to treat the same (or a different) area of the head scalp, during the next treatment sessions according to step 10H. In some embodiments, the treatment method is repeated 3-6 times with 3-week interval between the consecutive treatments. In some embodiments, the method comprises performing each treatment session for about 15 to 60 minutes. In some embodiments, the method comprises applying the treatment in a stamping mode by contacting the treatment site with the handpiece, activating the laser module for a predetermined period, stopping the laser module, moving the handpiece to a next treatment site and starting again.

Optionally, during the treatment, the method comprises cooling the treatment site to keep it at optimal temperature while enabling increasing at least one of the energy per micro-beam and micro-beam density of the fractional pattern, which otherwise are unbearable, and while preventing adverse effects, burns or scars, that might be caused by the intensive treatment. Cooling the treatment site can be done passively by contacting the treatment site with a tip being actively cooled (e.g., by the first cooling device described above), and/or actively by applying direct cooling, e.g. by cooled air, to the treatment site. In some embodiments, the method comprises providing cooling to the treatment site by the treatment window, the hair separator and/or the tail of the tip end, either by passively cooling the treatment site or by actively cooling the treatment window, the hair separator and/or the tail of the tip end and transferring the cooling to the treatment site.

The applicant has performed animal studies as well as clinical studies to evaluate hair growth (e.g., as demonstrated through improvement in scalp hair appearance) in males and females utilizing treatment based on the presently disclosed subject matter.

In one clinical study, the study included one hundred and thirty-two (132) participants, with ages ranging from 21 to 80 years and a mean age of 38.2±11.5 years. Of them, in 57 (57/132, 43.2%) of the participants the documented diagnosis was Androgenic Alopecia, and in 75 participants (75/132, 56.8%) there was no documented diagnosis in the clinic records. The number of males and females' participants was identical (66), and the majority of subjects had Fitzpatrick skin type III (79/132, 59.8%)), with some having skin types II (26/132, 19.7%) or IV (3/132, 2.3%). Skin type was not specified for 24 subjects (24/132, 18.2%).

A total of ninety-eight (98) cases were evaluated in another study, with ages ranging from 21 to 66 years old and a mean age of 37.2±9.9 years. Of them, in 44 (44/98, 44.9%) of the cases the documented diagnosis was Androgenic Alopecia, and in 54 cases (54/98, 55.1%) there was no documented diagnosis. Females slightly outnumbered males (51 vs. 47), and the majority of subjects had Fitzpatrick skin type III (60/98, 61.2%), with some having skin types II (18/98, 18.4%) or IV (1/98, 1%). Skin type was not specified for 19 subjects. Fourteen subjects had an endocrine medical disorders. Four subjects were taking concomitant medications, and only one subject had undergone previous hair growth treatment.

In the study, all hair growth treatments utilized fractional non-ablative laser beams of 1565 nm wavelength, the density of spots per square centimeter was 350, and the energy levels ranged from 17 to 19 mJ.

The majority of patients underwent either 3 (74/132, 56.1%) or 4 (39/132, 29.5%) treatments, while a smaller number received 5 (9/132, 6.8%) or 6 (10/132, 7.6%) treatments.

No adverse reactions were reported among the 132 enrolled participants in the first study during or following the laser treatment, indicating the safety of the treatment. The primary performance endpoint was evaluated by measuring the rate of accurate identification of the post treatment images by 3 blinded reviewers. Success was defined as correct identification of the post-treatment image by two or more blinded reviewers. For the 98 included subjects, the overall success rate was 96.9% (95% CI: 91.38%-98.95%). Among the sub-groups, the success rates were 97.7% (95% CI: 87.8%-99.7%) for subjects with Androgenic Alopecia (n=44), 96.3% (95% CI: 84.7%-99.2%) for those with no documented diagnosis (n=54), 97.9% (95% CI: 91.1%-99.7%) for male subjects (n=47), and 96.1% (95% CI: 82.5%-99.2%) for female subjects (n=51).

Based on the data presented in the retrospective, observational, single-center study, it can be concluded that the use of fractional non-ablative 1565 nm laser beams is safe and effective in promoting visible hair growth and improving scalp hair appearance. The effectiveness of the treatment was statistically significant, as demonstrated by the high percentage of cases classified correctly by blinded reviewers. Therefore, the findings suggest that fractional non-ablative 1565 nm laser treatment can be considered as a viable option for individuals seeking to improve hair growth.

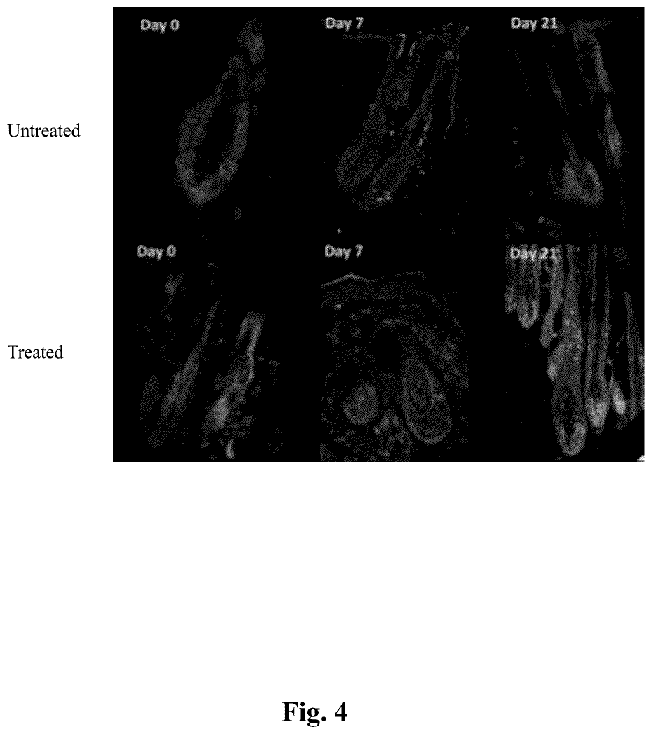

In an in-vivo animal study on mice, the applicant has found that a fractional non-ablative 1565 nm laser treatment is effective for the regeneration of hair follicles. The study included gene expression analysis of Cytokine's expression indicative of inflammation, which was significantly enhanced during the treatment. The study included histological analysis which showed the fractional non-ablative laser effect of tissue coagulation at day 0, infiltration of immune factors at day 7 and complete tissue repair at day 21 with the observation of complete formation of hair follicles. Immunofluorescence images illustrating the regeneration of the hair follicles under treatment carried out with the presently disclosed subject matter are shown in FIG. 4.

Various detailed embodiments of the present disclosure, taken in conjunction with the accompanying figures, are disclosed herein; however, it is to be understood that the disclosed embodiments are merely illustrative. The aforementioned examples are, of course, illustrative and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure. In addition, throughout the specification, the meaning of “a,” “an,” and “the” include plural references. The meaning of “in” includes “in” and “on.”

1. A system for hair treatment comprising:
a laser module configured for generating laser light;
a handpiece connected to the laser module, configured to receive the laser light and deliver a fractional pattern of non-ablative laser beams to a treatment site on a person's scalp;
one more of tips, each comprising a treatment window, configured to be mounted at a distal end of the handpiece;
a controller adapted for controlling parameters of the non-ablative laser beams to apply to the person's scalp;
a non-transitory computer readable medium programmed with computer readable code that upon execution by the controller causes the controller to:
activate the laser module for a predetermined treatment time, at a predetermined pulse rate and a predetermined pulse duration;
deliver the non-ablative laser beams to the treatment site on the person's scalp; and
deactivate the laser module.

2. The system of claim 1, wherein the treatment window is made from sapphire.

3. The system of claim 1, wherein the treatment window has a shape of at least one of the following:
a polygonal shape;
a non-regular shape;
a half polygon shape;
a half circle shape;
a circle shape:
a half elliptical shape;
an elliptical shape; and
a U shape.

4. The system of claim 1, wherein the tip comprises a visualization window configured to provide visualization of the treatment site and/or the treatment window.

5. the system of claim 4, wherein the visualization window is a hole or opening in a wall of the tip.

6. The system of claim 4, wherein the tip comprises a plurality of visualization windows with at least one of a front side orientation and a back side orientation, in relation to a treatment direction.

7. The system of claim 1, wherein the tip is either:
a disposable tip configured for a single use or a single patient; or
a reusable tip adapted for sterilization between treatments.

8. The system of claim 1, wherein at least one of the one or more tips further comprises an elongated bar forming a spacer defined by a distance between the laser output and the treatment site.

9. The system of claim 8, wherein the spacer is at least one of: an integral part of the tip or a connectable spacer to a distal end of the tip.

10. The system of claim 1, wherein the one or more tips are configured to be mounted at a distal end of the handpiece, and the tips are configured to do at least one of the following:
define the shape of the treatment site;
form a spacer between a laser output of the handpiece and the treatment site;
move grown hair aside for treatment; or
conform to topography of the treatment site.

11. A method for hair treatment comprising:
providing a laser module configured for generating laser light;
providing a handpiece connected to the laser module configured to receive the laser light and deliver a fractional pattern of non-ablative laser beams to a treatment site on a person's scalp;
providing one more of tips, each comprising a treatment window, configured to be mounted at a distal end of the handpiece;
providing a controller adapted for controlling parameters of the non-ablative laser beams to apply the scalp;
activating, by the controller, the laser module for a predetermined treatment time, at a predetermined pulse rate and a predetermined pulse duration;
delivering, by the handpiece, through the treatment window, the non-ablative laser beams to the treatment site on the person's scalp; and
deactivating, by the controller, the laser module.

12. The method of claim 11 wherein the treatment window is made from sapphire.

13. The method of claim 11, wherein the tip comprises a visualization window configured to provide visualization of the treatment site and/or the treatment window.

14. The method of claim 13, wherein the visualization window is a hole or opening in a wall of the tip.

15. the method of claim 13, wherein the tip comprises a plurality of visualization windows with at least one of a front side orientation and a back side orientation in relation to a treatment direction.

16. The method of claim 11, wherein at least one of the one or more tips further comprises an elongated bar forming a spacer defined by a distance between the laser output and the treatment site.

17. The method of claim 16, wherein the spacer is at least one of an integral part of the tip or a connectable spacer to a distal part of the tip.

18. The method of claim 11, wherein the delivering, by the handpiece, of the non-ablative laser beams to the treatment site on the person's scalp is done by sliding or gliding the tip over the treatment of the person's scalp.
